(19) United States
(12) Patent Application Publication
(10) Pub. No.: US 2024/0335563 A1
(43) Pub. Date: Oct. 10, 2024

(54) EPIGENETIC MODULATORS FOR TISSUE REPROGRAMMING

(71) Applicant: THE TRUSTEES OF INDIANA UNIVERSITY, Bloomington, IN (US)

(72) Inventors: Chandan K. SEN, Indianapolis, IN (US); Manishekhar KUMAR, Indianapolis, IN (US); Kanhaiya SINGH, Indianapolis, IN (US); Sashwati ROY, Indianapolis, IN (US)

(21) Appl. No.: 18/294,722

(22) PCT Filed: Aug. 11, 2022
(86) PCT No.: PCT/US2022/074811

§ 371 Date: Feb. 2, 2024

Related U.S. Application Data
(60) Provisional application No. 63/231,780, filed on Aug. 11, 2021.

(51) Int. Cl. A61K 48/00; A61K 31/7068; A61P 3/10; C12N 9/02; C12N 9/22; C12N 15/11; C12N 15/90

(52) U.S. Cl. CPC A61K 48/0058; A61K 31/7068; A61P 3/10; C12N 9/0071; C12N 9/22; C12N 15/11; C12N 15/90; C12Y 114/11; C12N 2310/20

(57) ABSTRACT

Disclosed herein are compositions and in vitro and in vivo methods for reducing diabetes-induced epigenetic barriers in cells to enhance the efficacy of treatments of diabetes.

### CROSS REFERENCE TO RELATED APPLICATIONS

This application claims priority to U.S. Provisional Patent Application Nos. 63/231,780 filed on Aug. 11, 2021, the disclosure of which is expressly incorporated herein.

### INCORPORATION BY REFERENCES OF MATERIAL SUBMITTED ELECTRONICALLY

Incorporated by reference in its entirety is a computer-readable nucleotide/amino acid sequence listing submitted concurrently herewith and identified as follows: 17 kilobytes xml file named “83677-35806.xml” created on Aug. 8, 2022.

### BACKGROUND

In the United States, 2% of the population are affected by chronic wounds. Chronic wounds do not follow the normal process of healing and remain open for more than a month. Because of sharp escalations in health care cost burden, rapidly growing aging population and rapid rise in the incidence of diabetes and obesity, the burden of treating chronic wounds is growing rapidly. Molecular analyses of the wound tissue of patients suffering from chronic wounds represents a powerful approach to identify clinically relevant therapeutic targets.

DNA methylation is known to regulate cell proliferation, cell migration, and cell differentiation. While studies of the heart and kidney have shown that ischemia can be a potent inducer of gene methylation, and it is widely known that ischemia is a common complication factor underlying chronic wound closure, the full significance of gene methylation in wound healing remains to be addressed.

Diabetes conditions are known to present a barrier to therapeutic tissue reprogramming and to impede wound healing. As disclosed herein for the first time, applicant provides evidence that epigenetic alterations induced by diabetic conditions play a role in diminishing therapeutic tissue reprogramming and impeding wound healing in patients.

### SUMMARY

Under condition of diabetes, epigenetic silencing of genetic pathways compromise molecular mechanisms necessary to achieve therapeutic tissue reprogramming. As disclosed herein, diabetes conditions pose a barrier to therapeutic tissue reprogramming that applicant has demonstrated is caused by epigenetic interference. In accordance with one embodiment of the present invention, approaches to manage diabetes-induced epigenetic barriers are provided as a novel approach to improved therapeutic outcomes in subjects suffering from chronic wounds. This principle applies to all forms of tissue reprogramming across all tissue systems in diabetic subjects, including patients with type 2 diabetes. Approaches to manage such a diabetic epigenetic barrier may include drugs, gene transfer, gene editing, and other strategies.

In accordance with one embodiment a method of normalizing blood glucose levels in a subject with diabetes is provided, wherein diabetes-induced epigenetic barriers in cells are reduced by introducing one or more epigenetic modulators into said cells, including for example skin tissue cells. In one embodiment the epigenetic modulator is an inhibitor of methyltransferases, including for example 5-Azacytidine.

In one embodiment the epigenetic modulator is targeted to specific genes for modulation of epigenic markers on the target gene. More particularly, in one embodiment the epigenetic modulator comprises a dCas9-TET1CD system for targeted demethylation of genomic regions. In this embodiment the promoter of a target gene can be demethylated through the use of a gene targeting guide RNA and a polynucleotide encoding a dCas9-TET1CD fusion peptide, after the guide RNA and polynucleotide encoding a dCas9-TET1CD fusion peptide are co-transfected into tissues of a subject in need thereof.

In one embodiment the method of normalizing blood glucose levels in a subject with diabetes, including type 2 diabetes, comprises the step of reprogramming targeted skin tissue in vivo to produce insulin by contacting the cells of said target skin tissue with an epigenetic modulator composition and with a reprogramming composition under conditions that enhance cellular uptake of the reprogramming composition components. In one embodiment, wherein the reprogramming composition comprises
a first nucleic acid sequence encoding a peptide having at least 95% sequence identity to SEQ ID NO: 2;
a second nucleic acid sequence encoding a peptide having at least 95% sequence identity to SEQ ID NO: 4; and
a third nucleic acid sequence encoding a peptide having at least 95% sequence identity to SEQ ID NO: 6; and optionally
a fourth nucleic acid sequence encoding a peptide having at least 95% sequence identity to SEQ ID NO: 8 to said target skin cells.

In one embodiment a diabetic subject is administered an epigenetic modulator in conjunction with a therapeutic agent for treating diabetes, including type 2 diabetes. For example, the therapeutic agent for treating diabetes can be selected from any of the known ten classes of orally available pharmacological agents to treat type 2 diabetes: 1) sulfonylureas, 2) meglitinides, 3) metformin (a biguanide), 4) thiazolidinediones (TZDs), 5) alpha glucosidase inhibitors, 6) dipeptidyl peptidase IV (DPP-4) inhibitors, 7) bile acid sequestrants, 8) dopamine agonists, 9) sodium-glucose transport protein 2 (SGLT2) inhibitors and 10) oral glucagon like peptide 1 (GLP-1) receptor agonists. In one embodiment the therapeutic agent is a reprogramming composition as described herein.

### BRIEF DESCRIPTION OF THE DRAWINGS

FIG. 1 presents data collected from 25 streptozotocin-induced diabetic C57BL/6 male mice. Control, n=9, TNT_{PMGF}, n=16. Starting mouse age: 8-10 weeks. PDX-1, MafA, GLP-1R and FGF21 based “PMGF” cocktail was delivered to these mice topically

via Tissue Nano transfection (TNT) technique.

FIG. 2: One-way ANOVA, Tukey's test compared to TNTₛₕₐₘ

FIG. 3 is a graph showing data from 09 streptozotocin-induced diabetic C57BL/6 male mice. Two weeks post-TNT_{PMGF} intervention these mice were not responding to the transfection treatment (average blood glucose˜400 mg/dL, week 4). To investigate the role of epigenetic factors in the failure of the TNT_{PMGF} intervention, an epigenetic modulator (drug 5-Azacytidine in this case) was applied at TNT sites. The drug was injected intradermally (10 mg/Kg body weight) thrice a week (at week 4) on alternate days (day 1, 3 and 5). Injections of epigenetic modulator applied to these mice significantly lowered the blood glucose level (average blood glucose˜275 mg/dL, week 5; p<0.001 vs. week 4) in all the non-responding mice, thus converting them to responders. Subsequent 5-Azacytidine injection (at week 7) could maintain the blood glucose levels in normoglycemic range (˜170 mg/dL, week 8; p<0.001 and 169 mg/dL, week 9; p<0.001 vs. week 4).

FIGS. 4A-4C: Targeted demethylation in keratinocytes using CRISPR-Cas9 and a peptide-repeat-based amplification. Representation of vector components used in the experiment are shown in FIG. 4A. dCas9 fused to a peptide repeat can recruit multiple copies of antibody (scFv)-fused TET1CD. Hence, multiple copies of TET1CD can demethylate the target more efficiently. Keratinocyte targeting was achieved by KRT14 promoter driven expression of guide RNAs. The mouse TP53 promoter locus used for demethylation events. The locations of the targets (1-3) for sgRNAs are indicated by pointers. FIG. 4B provides a schematic diagram of the TNT process on murine bipedicle wound. The positive electrode is inserted intradermally, and the negative electrode is put in contact with the cargo solution. A pulsed electric field (250 V, 10 ms pulses, 10 pulses) is then applied across the electrodes to nanoporate exposed cell membranes and inject the cargo directly into the cytosol. FIG. 4C shows the flow validation of keratinocyte specific delivery of gene editing cocktail. 70.9±3.1% KRT14+ cells were also expressing GFP (n=6 different skin sites from 3 animals).

FIG. 5A Schematic diagram showing the timeline of topical delivery of TET1 CD and targeted sgRNAs to the ischemic bipedicle wound created on dorsal skin of C57BL/6 mice through tissue nanotransfection technology (TNT). FIG. 5B: Wound closure was monitored at different days post-wounding in bipedicle ischemic wounds of mice nanotransfected with TET1CD and peptide repeat in presence or absence of KRT14 promoter driven gRNA targets by digital planimetry (left). Data was presented as percentage of wound area (right). n=8, *P<0.05 (Student's t test). FIG. 5C: Wound epithelization was monitored at different days post-wounding in bipedicle ischemic wounds of mice nanotransfected with TET1CD and peptide repeat in presence or absence of KRT14 promoter driven gRNA targets by hematoxylin and eosin staining (left). Data was presented as non-epithelized wound length (right). n=8, *P<0.05 (Student's t test).

### DETAILED DESCRIPTION

### Definitions

In describing and claiming the invention, the following terminology will be used in accordance with the definitions set forth below.

The term “about” as used herein means greater or lesser than the value or range of values stated by 10 percent but is not intended to limit any value or range of values to only this broader definition. Each value or range of values preceded by the term “about” is also intended to encompass the embodiment of the stated absolute value or range of values.

As used herein, the term “purified” and like terms relate to the isolation of a molecule or compound in a form that is substantially free of contaminants normally associated with the molecule or compound in a native or natural environment. As used herein, the term “purified” does not require absolute purity; rather, it is intended as a relative definition. The term “purified polypeptide” is used herein to describe a polypeptide which has been separated from other compounds including, but not limited to nucleic acid molecules, lipids and carbohydrates.

The term “isolated” requires that the referenced material be removed from its original environment (e.g., the natural environment if it is naturally occurring). For example, a naturally-occurring polynucleotide present in a living animal is not isolated, but the same polynucleotide, separated from some or all of the coexisting materials in the natural system, is isolated.

Tissue nanotransfection (TNT) is an electroporation-based technique capable of delivering nucleic acid sequences and proteins into the cytosol of cells at nanoscale. More particularly, TNT uses a highly intense and focused electric field through arrayed nanochannels, which benignly nanoporates the juxtaposing tissue cell members, and electrophoretically drives cargo (e.g., nucleic acids or proteins) into the cells.

As used herein a “control element” or “regulatory sequence” are non-translated regions of a functional gene, including enhancers, promoters, 5′ and 3′ untranslated regions, which interact with host cellular proteins to carry out transcription and translation. Such elements may vary in their strength and specificity. “Eukaryotic regulatory sequences” are non-translated regions of a functional gene, including enhancers, promoters, 5′ and 3′ untranslated regions, which interact with host cellular proteins of a eukaryotic cell to carry out transcription and translation in a eukaryotic cell including mammalian cells.

As used herein a “promoter” is a sequence or sequences of DNA that function when in a relatively fixed location in regard to the transcription start site of a gene. A “promoter” contains core elements required for basic interaction of RNA polymerase and transcription factors and can contain upstream elements and response elements. As used herein an “Enhancer” is a sequence of DNA that functions independent of distance from the transcription start site and can be either 5′ or 3′ to the transcription unit. Furthermore, enhancers can be within an intron as well as within the coding sequence itself. They are usually between 10 and 300 bp in length, and they function in cis. Enhancers function to increase transcription from nearby promoters. Enhancers, like promoters, also often contain response elements that mediate the regulation of transcription. Enhancers often determine the regulation of expression.

The term “identity” as used herein relates to the similarity between two or more sequences. Identity is measured by dividing the number of identical residues by the total number of residues and multiplying the product by 100 to achieve a percentage. Thus, two copies of exactly the same sequence have 100% identity, whereas two sequences that have amino acid deletions, additions, or substitutions relative to one another have a lower degree of identity. Those skilled in the art will recognize that several computer programs, such as those that employ algorithms such as BLAST (Basic Local Alignment Search Tool, Altschul et al. (1993) J. Mol. Biol. 215:403-410) are available for determining sequence identity.

As used herein, the term “pharmaceutically acceptable carrier” includes any of the standard pharmaceutical carriers, such as a phosphate buffered saline solution, water, emulsions such as an oil/water or water/oil emulsion, and various types of wetting agents. The term also encompasses any of the agents approved by a regulatory agency of the US Federal government or listed in the US Pharmacopeia for use in animals, including humans.

As used herein, the term “treating” includes prophylaxis of the specific disorder or condition, or alleviation of the symptoms associated with a specific disorder or condition and/or preventing or eliminating said symptoms.

As used herein an “effective” amount or a “therapeutically effective amount” of a drug refers to a nontoxic but enough of the drug to provide the desired effect. The amount that is “effective” will vary from subject to subject or even within a subject overtime, depending on the age and general condition of the individual, mode of administration, and the like. Thus, it is not always possible to specify an exact “effective amount.” However, an appropriate “effective” amount in any individual case may be determined by one of ordinary skill in the art using routine experimentation.

As used herein the term “patient” without further designation is intended to encompass any warm-blooded vertebrate domesticated animal (including for example, but not limited to livestock, horses, cats, dogs and other pets) and humans receiving therapeutic care with or without physician oversight.

The term “carrier” means a compound, composition, substance, or structure that, when in combination with a compound or composition, aids or facilitates preparation, storage, administration, delivery, effectiveness, selectivity, or any other feature of the compound or composition for its intended use or purpose. For example, a carrier can be selected to minimize any degradation of the active ingredient and to minimize any adverse side effects in the subject.

The term “inhibit” refers to a decrease in an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

The term “vector” or “construct” designates a nucleic acid sequence capable of transporting into a cell another nucleic acid to which the vector sequence has been linked. The term “expression vector” includes any vector, (e.g., a plasmid, cosmid or phage chromosome) containing a gene construct in a form suitable for expression by a cell (e.g., linked to a transcriptional control element). “Plasmid” and “vector” are used interchangeably, as a plasmid is a commonly used form of vector. Moreover, the invention is intended to include other vectors which serve equivalent functions.

The term “operably linked to” refers to the functional relationship of a nucleic acid with another nucleic acid sequence. Promoters, enhancers, transcriptional and translational stop sites, and other signal sequences are examples of nucleic acid sequences that can operably linked to other sequences. For example, operable linkage of DNA to a transcriptional control element refers to the physical and functional relationship between the DNA and promoter such that the transcription of such DNA is initiated from the promoter by an RNA polymerase that specifically recognizes, binds to and transcribes the DNA.

### EMBODIMENTS

Somatic cell reprogramming (SCR) involves massive reconfiguration of chromatin structure, from DNA methylation, to histone modifications, to nucleosome remodeling. These present themselves as ‘epigenetic barriers’ during reprogramming, as they are generally used as repressive mechanisms in somatic cells to prevent unwanted gene expression from other lineages. The use of epigenetic inhibitors (modifiers) demonstrates the importance in reprogramming the epigenome. For example, inhibition of DNA methylation with the DNA methyltransferase (DNMT) inhibitor 5-aza-cytidine (5-AZA) during reprogramming allows intermediate iPSCs to be fully reprogrammed. Inhibition of histone deacetylation using the HDAC inhibitors valproic acid (VPA) and trichostatin A (TSA) also improves SCR efficiency. In fact, treatment with TSA alone (without transduction of the reprogramming factors) is sufficient to upregulate ESC-specific genes. The use of Parnate (tranylcypromine), an inhibitor of LSD1, also increases the reprogramming efficiency of mouse fibroblasts. Finally, induction of histone H3K9 hypomethylation using a G9a methyltransferase chemical inhibitor BIX-01294, enhances the reprogramming of neural precursor cells and MEFs into iPSCs. PMID: 21621281.

Thus, the epigenetic modifications in specific gene regions lead to activation of associated genes and the intervention of these modifiers increases the reprogramming efficiency of non-reprogrammed/partially reprogrammed cells by resetting their epigenetic memory. As disclosed herein for the first time, diabetes conditions pose epigenetic barriers to therapeutic tissue reprogramming. Overcoming this barrier would result in efficient reprogramming in diabetic conditions including for example:
1. Vasculogenic reprogramming for rapid healing of chronic diabetic wounds,
2. Insulinogenic reprogramming to ameliorate hyperglycemia,
3. Neuronal reprogramming to overcome diabetic neuropathy and so on.

As disclosed herein compositions and methods are provided for reducing diabetes-induced epigenetic barriers in cells. The method comprises modifying, reducing or removing the methylation of genes involved in complications associated with diabetic patients. More particularly, the method comprises introducing an epigenetic modulator into said cells. In one embodiment the method comprises administering an epigenetic modulator to the cells of diabetic patients to treat chronic wounds, hyperglycemia or diabetic neuropathy. In one embodiment the administration of the epigenetic modulator is the single therapeutic agent used to treat the condition. In other embodiments the administration of the epigenetic modulator is used in conjunction with another known therapeutic strategy, wherein the administration of the epigenetic modulator reduces diabetes-induced epigenetic barriers and enhances the efficacy of the conventional diabetes treatment. In one embodiment the epigenetic modulator is an inhibitor of methyltransferases, including for example, 5-Azacytidine.

In one embodiment specific genes are targeted for demethylation through the use of a dCas9-TET1CD system (see Choudhury, et al, Oncotarget (Jul. 19, 2016); 7 (29): 46545-46556, the disclosure of which is incorporated herein). This construct consists of the catalytic domain of Ten-Eleven Translocation dioxygenase 1 (TET1CD) fused to the C-terminus of Cas9 Double Mutant (dCas9). The Cas9 Double Mutant has changes at amino acid positions D10A and H840A which completely inactivate both nuclease and nickase activities; whereas the TET1 domain facilitates the process of demethylation leading to transcriptional up-regulation. By using specific sgRNAs, the dCas9-TET1CD can be targeted to a promoter region allowing for epigenetic control over the expression of almost any gene of interest. The use of this system can target genes whose expression has been negatively impacted by diabetes-induced epigenetic modifications.

The epigenetic modulator polynucleotides of the present disclosure may be delivered to target tissues using any standard technique, including via a gene gun, a microparticle or nanoparticle suitable for such delivery, a liposome or other membrane bound vesicle suitable for such delivery, injection of DNA or viral-based vectors, or transfection by electroporation, using a three-dimensional nanochannel electroporation, a tissue nanotransfection (TNT) device, or a deep-topical tissue nanoelectroinjection device. In some embodiments, a viral vector can be used. However, in other embodiments, the polynucleotides are not delivered virally.

Electroporation is a technique in which an electrical field is applied to cells in order to increase permeability of the cell membrane, allowing cargo (e.g., reprogramming factors) to be introduced into cells. Electroporation is a common technique for introducing foreign DNA into cells. Additional details regarding such devices have been described in published International Application no. WO2021/016074, the disclosure of which is expressly incorporated by reference.

Tissue nanotransfection allows for direct cytosolic delivery of cargo (e.g, reprogramming factors) into cells by applying a highly intense and focused electric field through arrayed nanochannels, which benignly nanoporates the juxtaposing tissue cell members, and electrophoretically drives cargo into the cells.

In accordance with one embodiment tissue nanotransfection is used to deliver an epigenetic modulator into tissues adjacent or bordering a chronic would to modify, reduce or remove the methylation of target genes. The target genes are selected for demethylation to enhance expression of the target genes and remove or diminish barriers to therapeutic tissue reprogramming and to enhance wound healing in diabetic patients. In one embodiment the tissues associated with a chronic wound are transfected with nucleic acids encoding a TET1CD-dCas9 fusion proteins. In one embodiment TET1CD-dCas9 encoding nucleic acids and sequence-specific sgRNAs are co-transfected into chronic wound associated tissues to enhance wound healing. In accordance with one embodiment the encoded TET1CD-dCas9 fusion proteins target one or more genes selected from the group consisting of Notch1, P53, ADAM17, Etv2, Foxc2, Fli1, Pdx1, MafA, Glp1r and Fgf21.

The compositions of the present invention may further comprise a pharmaceutical carrier. Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

TNT provides a method for localized gene delivery that causes direct conversion of tissue function in vivo under immune surveillance without the need for any laboratory procedures. By using TNT with plasmids, it is possible to temporally and spatially control overexpression of a gene. Spatial control with TNT allows for transfection of a target area such as a portion of skin tissue without transfection of other tissues.

In accordance with one embodiment, an improved method of normalizing blood glucose levels in a subject with diabetes is provided. The method comprises the step of reprogramming targeted skin tissue in vivo to produce insulin by:
contacting the cells of said target skin tissue with an epigenetic modulator composition;
contacting the cells of said target skin tissue with a reprogramming composition under conditions that enhance cellular uptake of the reprogramming composition components, wherein the reprogramming composition comprises
   a first nucleic acid sequence encoding a peptide having at least 95% sequence identity to SEQ ID NO: 2;
   a second nucleic acid sequence encoding a peptide having at least 95% sequence identity to SEQ ID NO: 4; and
   a third nucleic acid sequence encoding a peptide having at least 95% sequence identity to SEQ ID NO: 6; and
   a fourth nucleic acid sequence encoding a peptide having at least 95% sequence identity to SEQ ID NO: 8 to said target skin cells.

In one embodiment the epigenetic modulator composition comprises 5-Azacytidine. In another embodiment the epigenetic modulator composition comprises one or more dCas9-TET1CD constructs that target specific genomic regions for demethylation. In one embodiment the reprogramming composition is introduced into skin cells of skin via tissue nanotransfection and the epigenetic modulator composition is injected intradermally at the nanotransfection site. In one embodiment the reprogramming composition and the epigenetic modulator composition are both introduced into skin cells of skin via tissue nanotransfection.

In one embodiment a method for enhancing wound repair in diabetes patients is provided. The method comprises the step of administering a demethylation cocktail that targets demethylation of genes in cells involved in tissue remodeling and wound repair. In one embodiment the demethylation cocktail is targeted for delivery to keratinocytes. In one embodiment the demethylation cocktail comprises one or more dCas9-TET1CD constructs that target specific genomic regions for demethylation. In one embodiment the TP53 gene of keratinocytes is targeted for demethylation, optionally wherein the promoter of TP53 is targeted for demethylation using the dCas9-TET1CD system with a TP53 specific guide RNA. In one embodiment any of the demethylation cocktail disclosed herein is introduced into said cells via tissue nanotransfection.

In accordance with embodiment 1, a method of reducing diabetes-induced epigenetic barriers in cells is provided, wherein said method comprises introducing an epigenetic modulator into said cells.

In accordance with embodiment 2, the method of embodiment 1 is provided wherein the reduction in diabetes-induced epigenetic barriers is used to treat a condition selected from chronic wounds, hyperglycemia and diabetic neuropathy.

In accordance with embodiment 3, the method of embodiment 1 or 2 is provided
wherein a diabetic subject is administered an epigenetic modulator in conjunction with a therapeutic agent for treating diabetes.

In accordance with embodiment 4, the method of any one of embodiments 1 to 3 is provided wherein the epigenetic modulator is an inhibitor of methyltransferases.

In accordance with embodiment 5, the method of any one of embodiments 1 to 4 is provided wherein the epigenetic modulator is 5-Azacytidine.

In accordance with embodiment 6, the method of any one of embodiments 1 to 5 is provided wherein the epigenetic modulator is targeted to specific genes for modulation of epigenic markers on the target gene.

In accordance with embodiment 7, the method of any one of embodiments 1 to 6 is provided wherein the epigenetic modulator comprises a dCas9-TET1CD system for targeted demethylation of genomic regions.

In accordance with embodiment 8, the method of any one of embodiments 1 to 7 is provided wherein the promoter of a target gene is demethylated through the use of a gene targeting guide RNA and a polynucleotide encoding a dCas9-TET1CD fusion peptide are co-transfected with into tissues of a diabetic patient.

In accordance with embodiment 9, the method of any one of embodiments 1 to 8 is provided wherein the gene targeting guide RNA and polynucleotide encoding a dCas9-TET1CD fusion peptide are co-transfected into tissues adjacent to a chronic wound.

In accordance with embodiment 10, the method of any one of embodiments 1 to 9 is provided wherein said tissues are transfected via nanotransfection.

In accordance with embodiment 11, an improved method of normalizing blood glucose levels in a subject with diabetes is provided, said method comprising the step of reprogramming targeted skin tissue in vivo to produce insulin, said method comprising
contacting the cells of said target skin tissue with an epigenetic modulator composition;
contacting the cells of said target skin tissue with a reprogramming composition under conditions that enhance cellular uptake of the reprogramming composition components, wherein the reprogramming composition comprises
   a first nucleic acid sequence encoding a peptide having at least 95% sequence identity to SEQ ID NO: 2;
   a second nucleic acid sequence encoding a peptide having at least 95% sequence identity to SEQ ID NO: 4; and
   a third nucleic acid sequence encoding a peptide having at least 95% sequence identity to SEQ ID NO: 6; and
   a fourth nucleic acid sequence encoding a peptide having at least 95% sequence identity to SEQ ID NO: 8 to said target skin cells.

In accordance with embodiment 12, the method of claim 11 is provided wherein the epigenetic modulator composition comprises 5-Azacytidine.

In accordance with embodiment 13, the method of any one of embodiments 11 to 12 is provided wherein the reprogramming composition is introduced into skin cells of skin via tissue nanotransfection and the epigenetic modulator composition is injected intradermally at the nanotransfection site.

In accordance with embodiment 14, a method for enhancing wound repair in diabetes patients is provided, said method comprising the step of administering to said patient a demethylation cocktail that targets demethylation of genes in cells involved in tissue remodeling and wound repair.

In accordance with embodiment 15, the method of claim 14 is provided wherein the cells are keratinocytes.

In accordance with embodiment 16, the method of any one of embodiments 14 to 15 is provided wherein the targeted gene is TP53, optionally wherein the promoter of TP53 is targeted for demethylation using the dCas9-TET1CD system with a TP53 specific guide RNA.

In accordance with embodiment 17, the method of any one of embodiments 14 to 16 is provided wherein the demethylation cocktail is introduced into said cells via tissue nanotransfection.

### Example 1

### Epigenetic Modulator (5-Azacytidine) Overcomes Diabetes-Induced Epigenetic Barriers to TNT-Mediated PMGF Therapy.

The use of a transfection cocktail comprising nucleic acid sequences encoding for Pancreatic And Duodenal Homeobox 1 (PDX-1), transcription factor MafA, glucagon-like peptide 1 receptor (GLP-1R); and Fibroblast growth factor 21 (FGF21) (the “PMGF” cocktail) for lowering blood glucose levels in streptozotocin (STZ) induced diabetic mice was previously reported in PCT/US2021/039083, the disclosure of which is incorporated herein.

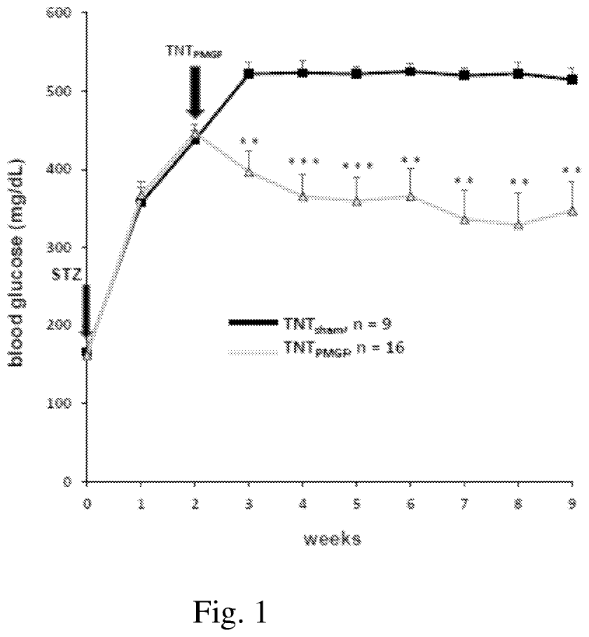

FIG. 1 presents data collected from 25 streptozotocin-induced diabetic C57BL/6 male mice. Control, n=9, TNT_{PMGF}, n=16. Starting mouse age: 8-10 weeks. PDX-1, MafA, GLP-1R and FGF21 based “PMGF” cocktail was delivered to these mice topically via Tissue Nano transfection (TNT) technique.

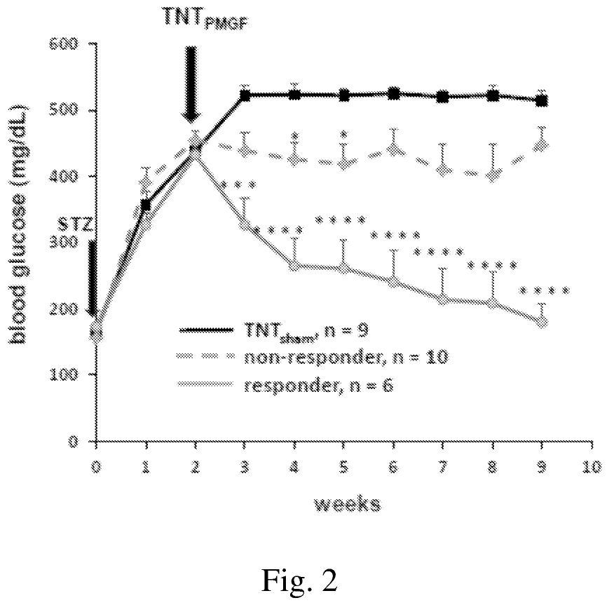

However subgroup analysis of the data from the 25 TNT_{PMGF} mice from the FIG. 1 group reveals a Responder (n=6) and a Non-responder (n=10) group. All male streptozotocin-induced diabetic C57BL/6 mice, starting mouse age 8-10 weeks. The term “Responders” identifies mice in which blood glucose level was measured to be below 200 mg/dL at any week time point whereas “Non-responders” retained elevated blood glucose levels. (See FIG. 2: One-way ANOVA, Tukey's test compared to TNTₛₕₐₘ).

### Epigenetic Modulator (5-Azacytidine) Overcomes Diabetes-Induced Epigenetic Barriers Resulting in Improved Therapeutic Outcomes.

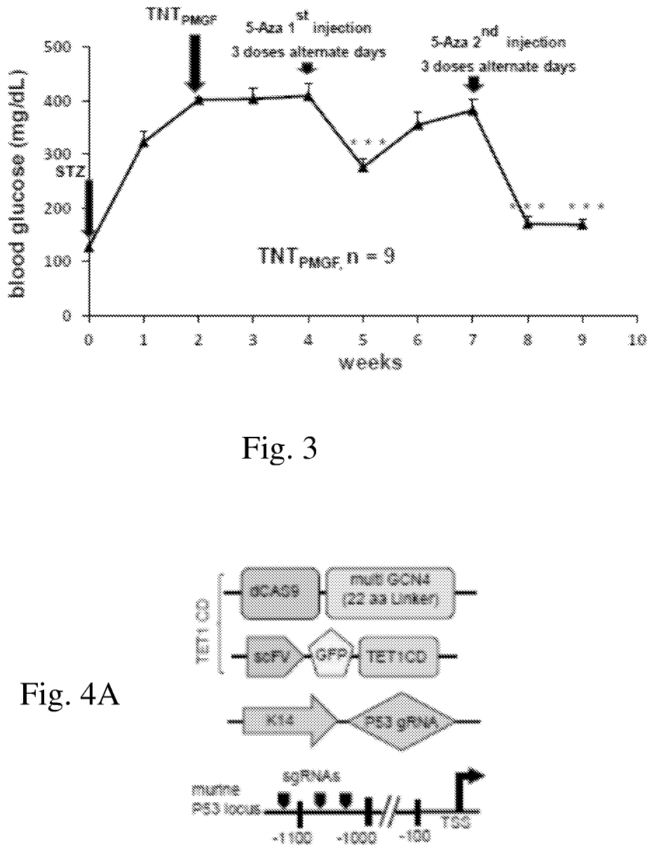

FIG. 3 is a graph showing data from 09 streptozotocin-induced diabetic C57BL/6 male mice. Two weeks post-TNT_{PMGF} intervention these mice were not responding to the transfection treatment (average blood glucose˜400 mg/dL, week 4). To investigate the role of epigenetic factors in the failure of the TNT_{PMGF} intervention, an epigenetic modulator (drug 5-Azacytidine in this case) was applied at TNT sites. The drug was injected intradermally (10 mg/Kg body weight) thrice a week (at week 4) on alternate days (day 1, 3 and 5). Injections of epigenetic modulator applied to these mice significantly lowered the blood glucose level (average blood glucose˜275 mg/dL, week 5; p<0.001 vs. week 4) in all the non-responding mice, thus converting them to responders. Subsequent 5-Azacytidine injection (at week 7) could maintain the blood glucose levels in normoglycemic range (˜170 mg/dL, week 8; p<0.001 and 169 mg/dL, week 9; p<0.001 vs. week 4).

### Example 2

### Targeted Demethylation of Specific Gene Promoters

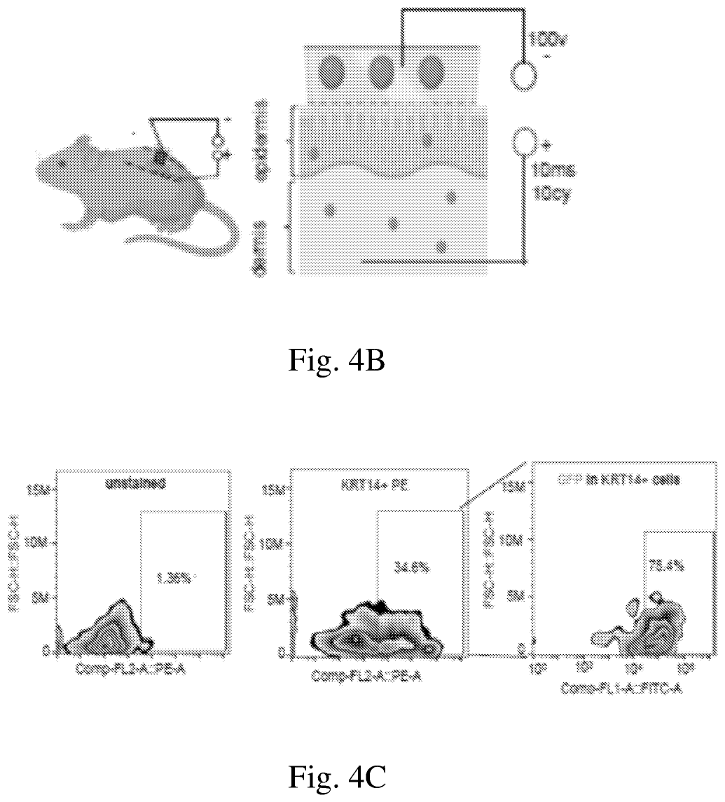

For TNT mediated targeted DNA demethylation of TP53 in murine ischemic wounds, we used a system in which an inactive Cas9 nuclease (dCas9) is fused with catalytic domain at the C terminus of TET1 (TET1 CD) (PMID: 27571369). This construct consists of the catalytic domain of Ten-Eleven Translocation dioxygenase 1 (TET1CD) fused to the C-terminus of Cas9 Double Mutant (dCas9). The Cas9 Double Mutant has changes at amino acid positions D10A and H840A which completely inactivate both nuclease and nickase activities; whereas the TET1 domain facilitates the process of demethylation leading to transcriptional up-regulation. By using specific sgRNAs, the dCas9-TET1CD can be targeted to a promoter region allowing for epigenetic control over the expression of almost any gene of interest. To increase the efficiency of targeted demethylation, we adopted the previously described dCas9-SUperNova Tagging (SunTag) with modified linker length to 22 amino acids (PMIDs: 25307933, 27571369, 29524149). See FIGS. 4A-4C which presents data from targeted demethylation in keratinocytes using CRISPR-Cas9 and a peptide-repeat-based amplification.

Representation of vector components used in the experiment are shown in FIG. 4A. dCas9 fused to a peptide repeat can recruit multiple copies of antibody (scFv)-fused TET1CD. Hence, multiple copies of TET1CD can demethylate the target more efficiently. Keratinocyte targeting was achieved by KRT14 promoter driven expression of guide RNAs. The mouse TP53 promoter locus used for demethylation events. The locations of the targets (1-3) for sgRNAs are indicated by pointers. FIG. 4B provides a schematic diagram of the TNT process on murine bipedicle wound. The positive electrode is inserted intradermally, and the negative electrode is put in contact with the cargo solution. A pulsed electric field (250 V, 10 ms pulses, 10 pulses) is then applied across the electrodes to nanoporate exposed cell membranes and inject the cargo directly into the cytosol. FIG. 4C shows the flow validation of keratinocyte specific delivery of gene editing cocktail. 70.9±3.1% KRT14+ cells were also expressing GFP (n=6 different skin sites from 3 animals).

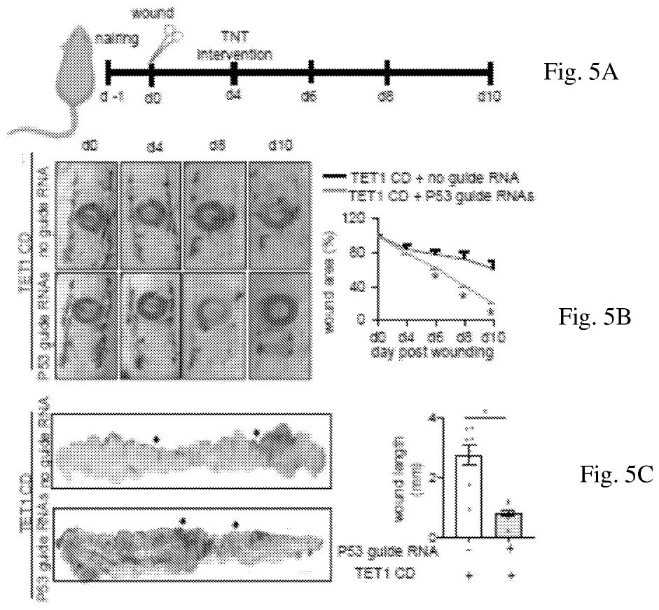

We applied this system to demethylate the promoter region of the TP53 gene in keratinocyte (KRT14+) compartment in murine ischemic bipedicle wounds using expression vectors for TP53 guide RNAs (gRNAs). We used our recently reported tissue TNT approach (see PCT/US2020/042510, the disclosure of which is incorporated herein) which allows direct cytosolic delivery of demethylation cocktail by applying a highly intense and focused electric field through arrayed nanochannels (PMIDs: 28785092, 32422219) which benignly nanoporates the juxtaposing tissue cell membranes and electrophoretically drives demethylation cocktail (FIG. 5A, 5B). First, the targeted delivery of guide RNAs to the keratinocytes was validated using flow cytometry (FIG. 5C). Subsequently the demethylation of the TP53 promoter was confirmed in murine ischemic wounds transfected with dCas9-TET1 CD complex and keratinocyte specific guide RNAs (FIG. 5C). Such demethylation of TP53 promoter was able to promote wound closure in murine ischemic wounds.

1. A method of reducing diabetes-induced epigenetic barriers in cells, said method comprising introducing an epigenetic modulator into said cells.

2. The method of claim 1 wherein the reduction in diabetes-induced epigenetic barriers is used to treat a condition selected from chronic wounds, hyperglycemia and diabetic neuropathy.

3. The method of claim 2 wherein a diabetic subject is administered an epigenetic modulator in conjunction with a therapeutic agent for treating diabetes.

4. The method of claim 1 wherein the epigenetic modulator is an inhibitor of methyltransferases.

5. The method of claim 4 wherein the epigenetic modulator is 5-Azacytidine.

6. The method of claim 1 wherein the epigenetic modulator is targeted to specific genes for modulation of epigenic markers on the target gene.

7. The method of claim 6 wherein the epigenetic modulator comprises a dCas9-TET1CD system for targeted demethylation of genomic regions.

8. The method of claim 7 wherein the promoter of a target gene is demethylated through the use of a gene targeting guide RNA and a polynucleotide encoding a dCas9-TET1CD fusion peptide are co-transfected with into tissues of a diabetic patient.

9. The method of claim 8 wherein the gene targeting guide RNA and polynucleotide encoding a dCas9-TET1CD fusion peptide are co-transfected into tissues adjacent to a chronic wound.

10. The method of claim 9 wherein said tissues are transfected via nanotransfection.

11. An improved method of normalizing blood glucose levels in a subject with diabetes, said method comprising the step of reprogramming targeted skin tissue in vivo to produce insulin, said method comprising
contacting the cells of said target skin tissue with an epigenetic modulator composition;
contacting the cells of said target skin tissue with a reprogramming composition under conditions that enhance cellular uptake of the reprogramming composition components, wherein the reprogramming composition comprises
a first nucleic acid sequence encoding a peptide having at least 95% sequence identity to SEQ ID NO: 2;
a second nucleic acid sequence encoding a peptide having at least 95% sequence identity to SEQ ID NO: 4; and
a third nucleic acid sequence encoding a peptide having at least 95% sequence identity to SEQ ID NO: 6; and
a fourth nucleic acid sequence encoding a peptide having at least 95% sequence identity to SEQ ID NO: 8 to said target skin cells.

12. The method of claim 11 wherein the epigenetic modulator composition comprises 5-Azacytidine.

13. The method of claim 12 wherein the reprogramming composition is introduced into skin cells of skin via tissue nanotransfection and the epigenetic modulator composition is injected intradermally at the nanotransfection site.

14. A method for enhancing wound repair in diabetes patients, said method comprising the step of administering a demethylation cocktail that targets demethylation of genes in cells involved in tissue remodeling and wound repair.

15. The method of claim 14 wherein the cells are keratinocytes.

16. The method of claim 15 wherein the targeted gene is TP53, optionally wherein the promoter of TP53 is targeted for demethylation using the dCas9-TET1CD system with a TP53 specific guide RNA.

17. The method of claim 16 wherein the demethylation cocktail is introduced into said cells via tissue nanotransfection.


SEQUENCE LISTING (8 sequences)

SEQ ID NO: 1 - DNA, 2603 bp, Homo sapiens
  source 1..2603: mol_type = other DNA
gagatcagtgcggagctgtcaaagcgagcaggggtggcgccgggagtgggaacgccacacagtgccaaatccccggctccagctcccgactcccggctcccggctcccggctcccggtgcccaatcccgggccgcagccatgaacggcgaggagcagtactacgcggccacgcagctttacaaggacccatgcgcgttccagcgaggcccggcgccggagttcagcgccagcccccctgcgtgcctgtacatgggccgccagcccccgccgccgccgccgcacccgttccctggcgccctgggcgcgctggagcagggcagccccccggacatctccccgtacgaggtgccccccctcgccgacgaccccgcggtggcgcaccttcaccaccacctcccggctcagctcgcgctcccccacccgcccgccgggcccttcccggagggagccgagccgggcgtcctggaggagcccaaccgcgtccagctgcctttcccatggatgaagtctaccaaagctcacgcgtggaaaggccagtgggcaggcggcgcctacgctgcggagccggaggagaacaagcggacgcgcacggcctacacgcgcgcacagctgctagagctggagaaggagttcctattcaacaagtacatctcacggccgcgccgggtggagctggctgtcatgttgaacttgaccgagagacacatcaagatctggttccaaaaccgccgcatgaagtggaaaaaggaggaggacaagaagcgcggcggcgggacagctgtcgggggtggcggggtcgcggagcctgagcaggactgcgccgtgacctccggcgaggagcttctggcgctgccgccgccgccgccccccggaggtgctgtgccgcccgctgcccccgttgccgcccgagagggccgcctgccgcctggccttagcgcgtcgccacagccctccagcgtcgcgcctcggcggccgcaggaaccacgatgagaggcaggagctgctcctggctgaggggcttcaaccactcgccgaggaggagcagagggcctaggaggaccccgggcgtggaccacccgccctggcagttgaatggggcggcaattgcggggcccaccttagaccgaaggggaaaacccgctctctcaggcgcatgtgccagttggggccccgcgggtagatgccggcaggccttccggaagaaaaagagccattggtttttgtagtattggggccctcttttagtgatactggattggcgttgtttgtggctgttgcgcacatccctgccctcctacagcactccaccttgggacctgtttagagaagccggctcttcaaagacaatggaaactgtaccatacacattggaaggctccctaacacacacagcggggaagctgggccgagtaccttaatctgccataaagccattcttactcgggcgacccctttaagtttagaaataattgaaaggaaatgtttgagttttcaaagatcccgtgaaattgatgccagtggaatacagtgagtcctcctcttcctcctcctcctcttccccctccccttcctcctcctcctcttcttttccctcctcttcctcttcctcctgctctcctttcctccccctcctcttttccctcctcttcctcttcctcctgctctcctttcctccccctcctctttctcctcctcctcctcttcttccccctcctctccctcctcctcttcttccccctcctctccctcctcctcttcttctccctcctcttcctcttcctcctcttccacgtgctctcctttcctccccctcctcttgctccccttcttccccgtcctcttcctcctcctcctcttcttctccctcctcttcctcctcctctttcttcctgacctctttctttctcctcctcctccttctacctccccttctcatccctcctcttcctcttctctagctgcacacttcactactgcacatcttataacttgcacccctttcttctgaggaagagaacatcttgcaaggcagggcgagcagcggcagggctggcttaggagcagtgcaagagtccctgtgctccagttccacactgctggcagggaaggcaaggggggacgggcctggatctgggggtgagggagaaagatggacccctgggtgaccactaaaccaaagatattcggaactttctatttaggatgtggacgtaattcctgttccgaggtagaggctgtgctgaagacaagcacagtggcctggtgcgccttggaaaccaacaactattcacgagccagtatgaccttcacatctttagaaattatgaaaacgtatgtgattggagggtttggaaaaccagttatcttatttaacattttaaaaattacctaacagttatttacaaacaggtctgtgcatcccaggtctgtcttcttttcaaggtctgggccttgtgctcgggttatgtttgtgggaaatgcttaataaatactgataatatgggaagagatgaaaactgattctcctcactttgtttcaaacctttctggcagtgggatgattcgaattcacttttaaaattaaattagcgtgttttgtttt

SEQ ID NO: 2 - protein, 283 aa, Homo sapiens
MNGEEQYYAATQLYKDPCAFQRGPAPEFSASPPACLYMGRQPPPPPPHPFPGALGALEQGSPPDISPYEVPPLADDPAVAHLHHHLPAQLALPHPPAGPFPEGAEPGVLEEPNRVQLPFPWMKSTKAHAWKGQWAGGAYAAEPEENKRTRTAYTRAQLLELEKEFLFNKYISRPRRVELAVMLNLTERHIKIWFQNRRMKWKKEEDKKRGGGTAVGGGGVAEPEQDCAVTSGEELLALPPPPPPGGAVPPAAPVAAREGRLPPGLSASPQPSSVAPRRPQEPR

SEQ ID NO: 3 - DNA, 2669 bp, Homo sapiens
  source 1..2669: mol_type = other DNA
agccgtgggaggcggggccggccggcggcgcgggtggggcgcgggagcggtcccggagcagcccgaggcggcggccgcggggaggaggcggcgacgcgggcccggggtcgcccgagacacctggccagcggtgcccctagcgcgccgccccggagttgaccacgtgaaacttttccctgcgcccctcggcgccgccgccccgcgccggcgcccccccgcccccgccgggaccgccgcccgcggggagcaggggggggagaggcctgcagctccccccccactcccacgccgcccgtcggggcgcggccgggcgcgggccccgggcgatggccgcggagctggcgatgggcgccgagctgcccagcagcccgctggccatcgagtacgtcaacgacttcgacctgatgaagttcgaggtgaagaaggagcctcccgaggccgagcgcttctgccaccgcctgccgccaggctcgctgtcctcgacgccgctcagcacgccctgctcctccgtgccctcctcgcccagcttctgcgcgcccagcccgggcaccggcggcggcggcggcgcggggggcggcggcggctcgtctcaggccgggggcgcccccgggccgccgagcgggggccccggcgccgtcgggggcacctcggggaagccggcgctggaggatctgtactggatgagcggctaccagcatcacctcaaccccgaggcgctcaacctgacgcccgaggacgcggtggaggcgctcatcggcagcggccaccacggcgcgcaccacggcgcgcaccacccggcggccgccgcagcctacgaggctttccgcggcccgggcttcgcgggcggcggcggagcggacgacatgggcgccggccaccaccacggcgcgcaccacgccgcccaccatcaccacgccgcccaccaccaccaccaccaccaccaccaccatggcggcgcgggacacggcggtggcgcgggccaccacgtgcgcctggaggagcgcttctccgacgaccagctggtgtccatgtcggtgcgcgagctgaaccggcagctccgcggcttcagcaaggaggaggtcatccggctcaagcagaagcggcgcacgctcaagaaccgcggctacgcgcagtcctgccgcttcaagcgggtgcagcagcggcacattctggagagcgagaagtgccaactccagagccaggtggagcagctgaagctggaggtggggcgcctggccaaagagcgggacctgtacaaggagaaatacgagaagctggcgggccggggcggccccgggagcgcgggcggggccggtttcccgcgggagccttcgccgccgcaggccggtcccggcggggccaagggcacggccgacttcttcctgtaggcgccggaccccgagcccgcgccgccgtcgccggggacaagttcgcgcaggcctctcggggcctcggctcggactccgcggtacaggacgtggacaccaggcccggcccggccgtgctggccccggtgccaagtctgcgggcgcggggctggaggccccttcgctcccggtccccgttcgcgcgcgtcggcccgggtcgccgtcctgaggttgagcggagaacggtgatttctaaggaaacttgagccaggtctaacttctttccaagcgtccgcttgtacatacgttgaacgtggttctccgttcccaccttcgccctgccagcctagagggaccgcgctgccgtcccttcccgggtggcccctgcctgcccccgccctccttcgttctcttctcagcctccctttccttgccttttttaacttcccctccccgttttaaaatcggtcttattttcgaagtatttataattattatgcttggtgattagaaaagaaaaccttggaggaagccccttctttccccagccggggtccgccctcagtcgcgagtcacagcatgagtcgctcgccaggaggggcccggcccctgcctgccccctccccgcttgcccccgaccctgctaccggcgttccttggaggtcgaagccagggacgtcacccgtgctgtgtccaggcctgctgtcctactatgctcaaccgggggtggggggaggggggtgagtcctgtgctcagtcgggtgggggctggcccggatcccgagctgctgtctctctatgcaccagaacatatctgtaactcctggggaaatacatcttgttttaaccttcaagagaagtgaaagaaaaaagtaatgcacagtatttctagcagaaaatttttttttttaagaggaggcttgggccagagccttctggcatggggcgggtggagaaagtgtttttattttaatttaaattgtgtttcgttttgtttgtggaatctttctttaatgcttcgtcgctctttggactagccgggagagagggcgaggaggcgggtgctccaggccctgtaggctgggccaggcgcctgggggatctgcccgttttcggaggccctcaggggccatcagtgggattccagccgctccacacccctcccctgagcactcggagtggaaggcgcgccgactcgttgaaagttttgttgtgtagttggttttcgttgagttcttttttcatttgctacgaaactgagaaaaagaaaaaaatacacaaaataaatctgttcagatccaa

SEQ ID NO: 4 - protein, 353 aa, Homo sapiens
MAAELAMGAELPSSPLAIEYVNDFDLMKFEVKKEPPEAERFCHRLPPGSLSSTPLSTPCSSVPSSPSFCAPSPGTGGGGGAGGGGGSSQAGGAPGPPSGGPGAVGGTSGKPALEDLYWMSGYQHHLNPEALNLTPEDAVEALIGSGHHGAHHGAHHPAAAAAYEAFRGPGFAGGGGADDMGAGHHHGAHHAAHHHHAAHHHHHHHHHHGGAGHGGGAGHHVRLEERFSDDQLVSMSVRELNRQLRGFSKEEVIRLKQKRRTLKNRGYAQSCRFKRVQQRHILESEKCQLQSQVEQLKLEVGRLAKERDLYKEKYEKLAGRGGPGSAGGAGFPREPSPPQAGPGGAKGTADFFL

SEQ ID NO: 5 - DNA, 1490 bp, Homo sapiens
  source 1..1490: mol_type = other DNA
gatggcccagtcctgaactccccgccatggccggcgcccccggcctgctgcgccttgcgctgctgctgctcgggatggtgggcagggccggcccccgcccccagggtgccactgtgtccctctgggagacggtgcagaaatggcgagaataccgacgccagtgccagcgctccctgactgaggatccacctcctgccacagacttgttctgcaaccggaccttcgatgaatacgcctgctggccagatggggagccaggctcgttcgtgaatgtcagctgcccctggtacctgccctgggccagcagtgtgccgcagggccacgtgtaccggttctgcacagctgaaggcctctggctgcagaaggacaactccagcctgccctggagggacttgtcggagtgcgaggagtccaagcgaggggagagaagctccccggaggagcagctcctgttcctctacatcatctacacggtgggctacgcactctccttctctgctctggttatcgcctctgcgatcctcctcggcttcagacacctgcactgcaccaggaactacatccacctgaacctgtttgcatccttcatcctgcgagcattgtccgtcttcatcaaggacgcagccctgaagtggatgtatagcacagccgcccagcagcaccagtgggatgggctcctctcctaccaggactctctgagctgccgcctggtgtttctgctcatgcagtactgtgtggcggccaattactactggctcttggtggagggcgtgtacctgtacacactgctggccttctcggtcttatctgagcaatggatcttcaggctctacgtgagcataggctggggtgttcccctgctgtttgttgtcccctggggcattgtcaagtacctctatgaggacgagggctgctggaccaggaactccaacatgaactactggctcattatccggctgcccattctctttgccattggggtgaacttcctcatctttgttcgggtcatctgcatcgtggtatccaaactgaaggccaatctcatgtgcaagacagacatcaaatgcagacttgccaagtccacgctgacactcatccccctgctggggactcatgaggtcatctttgcctttgtgatggacgagcacgcccgggggaccctgcgcttcatcaagctgtttacagagctctccttcacctccttccaggggctgatggtggccatattatactgctttgtcaacaatgaggtccagctggaatttcggaagagctgggagcgctggcggcttgagcacttgcacatccagagggacagcagcatgaagcccctcaagtgtcccaccagcagcctgagcagtggagccacggcgggcagcagcatgtacacagccacttgccaggcctcctgcagctgagactccagcgcctgccctccctggggtccttgctgcaggccgggtggccaatccaggtgggagagacactcc

SEQ ID NO: 6 - protein, 463 aa, Homo sapiens [sequence omitted]

SEQ ID NO: 7 - DNA, 1347 bp, Homo sapiens
  source 1..1347: mol_type = other DNA
acagatgaggttgaggttggcccacggccaggtgagaggcttccaaggcaggatacttgtgtctcagatgcggtcgcttctttcatacagcaattgccgccttgctgaggatcaaggaacctcagtgtcagatcacgccctccccccaaacttagaaattcagatggggcgcagaaatttctcttgttctgcgtgatctgcatagatggtccaagaggtggtttttccaggagcccagcacccctcctccctccgactcagacccaggagtctggccctccattgaaaggaccccaggttacatcatccattcaggctgcccttgccacgatggaattctgtagctcctgccaaatgggtcaaatatcatggttcaggcgcagggagggtgattgggcgggcctgtctgggtataaattctggagcttctgcatctatcccaaaaaacaagggtgttctgtcagctgaggatccagccgaaagaggagccaggcactcaggccacctgagtctactcacctggacaactggaatctggcaccaattctaaaccactcagcttctccgagctcacaccccggagatcacctgaggacccgagccattgatggactcggacgagaccgggttcgagcactcaggactgtgggtttctgtgctggctggtcttctgctgggagcctgccaggcacaccccatccctgactccagtcctctcctgcaattcgggggccaagtccggcagcggtacctctacacagatgatgcccagcagacagaagcccacctggagatcagggaggatgggacggtggggggcgctgctgaccagagccccgaaagtctcctgcagctgaaagccttgaagccgggagttattcaaatcttgggagtcaagacatccaggttcctgtgccagcggccagatggggccctgtatggatcgctccactttgaccctgaggcctgcagcttccgggagctgcttcttgaggacggatacaatgtttaccagtccgaagcccacggcctcccgctgcacctgccagggaacaagtccccacaccgggaccctgcaccccgaggaccagctcgcttcctgccactaccaggcctgccccccgcactcccggagccacccggaatcctggccccccagccccccgatgtgggctcctcggaccctctgagcatggtgggaccttcccagggccgaagccccagctacgcttcctgaagccagaggctgtttactatgacatctcctctttatttattaggttatttatcttatttatttttttatttttcttacttgagataataaagagttccagaggaggataa

SEQ ID NO: 8 - protein, 208 aa, Homo sapiens
MDSDETGFEHSGLWVSVLAGLLGACQAHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPALPEPPGILAPQPPDVGSSDPLSMVGPSQGRSPSYAS